(19) United States
(12) Patent Application Publication
(10) Pub. No.: US 2010/0120082 A1
(43) Pub. Date: May 13, 2010

(54) Optimization of Process Variables in Oxygen Enriched Fermentors Through Process Controls

(75) Inventors: Victor M. SAUCEDO, San Francisco, CA (US); Sudhir R. Brahmbhatt, Glencoe, MO (US)

Correspondence Address: AIR LIQUIDE;Intellectual Property, 2700 POST OAK BOULEVARD, SUITE 1800, HOUSTON, TX, 77056, US

(73) Assignee: American Air Liquide, Inc., Fremont, CA (US)

(21) Appl. No.: 12/689,838

(22) Filed: Jan. 19, 2010

Related U.S. Application Data
(63) Continuation of application No. 11/421,065, filed on May 30, 2006, now abandoned.
(60) Provisional application No. 60/686,730, filed on Jun. 2, 2005.

(51) Int. Cl. C12Q 1/02; C12M 1/34

(52) U.S. Cl. 435/29; 435/287.1

(57) ABSTRACT

Methods and systems are provided for controlling the addition of oxygen in fermentations to achieve a desired oxygen consumption and substrate yield in fermentation cell cultures. In one aspect, the invention provides a method for regulating the addition of oxygen (O₂) to a fermentor during a fermentation process, comprising measuring real-time dissolved oxygen (DO) in a fermentation broth, measuring real-time O₂ concentration in the fermentor exhaust, and providing the real-time DO measurement and real-time O₂ measurement to an adaptive controller configured to regulate O₂ flow into the fermentor responsive to the real-time DO measurement and real-time O₂ measurement.

### CROSS-REFERENCE TO RELATED APPLICATIONS

This application is a continuation of U.S. application Ser. No. 11/421,065, filed May 30, 2006, which claims the benefit under 35 U.S.C. §119(e) to provisional application No. 60/686,730, filed Jun. 2, 2005, the entire contents of which are incorporated herein by reference.

### BACKGROUND

1. Field of the Invention

The invention generally relates to the process productivity in fermentors, and more specifically to a system and method for controlling the addition of oxygen in fermentations to achieve an improved oxygen consumption and substrate yield in microorganisms.

2. Description of the Related Art

Biochemical engineering is a branch of chemical engineering which deals with the design and construction of unit processes involving microorganisms. Fermentation is one example of a process involving the bulk growth of microorganisms on a growth medium. Fermentation is one of the most important processes used in biochemical engineering, and the products of fermentations are extensively used in the pharmaceutical, biotechnology, brewing, and water treatment industries. Fermentations are typically conducted in a fermentor, or bioreactor, which may refer to any vessel or system that supports a biologically active environment. Industrial bioreactors can employ a variety of microorganisms, including bacteria and animal cells, ranging in complexity and response to sheer.

The design of a fermentation system is quite a complex engineering task. Under optimum conditions the microorganisms or cells are able to perform their desired function with great efficiency. The bioreactor's environmental conditions, such as gas (i.e., air, oxygen (O₂), nitrogen (N₂), carbon dioxide (CO₂)) flow rates, temperature, pH, dissolved oxygen levels, and/or agitation speed/circulation rate need to be closely monitored and controlled. To this end, most industrial bioreactor manufacturers use vessels, sensors, and controllers as components of fermentation systems.

Optimal oxygen transfer during a fermentation is perhaps the most difficult task to accomplish. Oxygen solubility in water is extremely low (at the parts per million level), and the solubility is even less in presence of solutes such as nutrients and other additions in broths. Oxygen is also only 20.9% by volume in air. Oxygen transfer in bioreactors can be enhanced by agitation in mechanical fermentors. Agitation is also needed to mix nutrients and to keep the fermentation homogeneous. However, there are limits to the speed of agitation, as it can induce high stress in organisms leading to cell death. High agitation speed also results in higher power consumption, increasing product unit costs. The dissolved oxygen (DO) in the growth media is usually measured to help determine the amount of oxidant gas that should be added to the fermentor.

Many different fermentation systems and their control of oxygen have been documented. One method attempts to improve the oxygen utilization in continuous fermentation of single cells by recycling fermentation liquid. In this approach either air, enriched oxygen, or pure oxygen is used during fermentations. However, the only control applied in this method is the level of liquid in the fermentor. Another method focuses specifically on one type of microorganism (Escherichia coli bacteria), but methods of controlling the oxygen supply are not provided. Instead, the method teaches the regulation of the carbon source as a function of the oxygen uptake rate of the microorganism. Yet another method describes a method of increasing the oxygen transfer in a fermentation system by introducing oxygen in only one portion of the broth that is sent back to the fermentor. Still another method describes a method of utilizing high pressure in the fermentor to promote oxygen dissolution and low pressure to remove CO₂. Still other methods teach a method of enriching bubble fermentors with oxygen while using air bubbles to agitate the growth media and eliminate CO₂ accumulated in the media.

However, the foregoing methods each fail to provide a method to regulate the real-time oxygen supply in agitation and bubble fermentors to improve oxygen utilization and maximize the productivity of the fermentation, leading to favorable system economics.

Therefore, there remains a need for a method to optimize the use of pure oxygen in fermentation systems to maximize productivity, substrate yield, and oxygen utilization of the fermentation cell culture.

### SUMMARY

Aspects of the invention generally provide a method for controlling the addition of oxygen in fermentations to achieve a desired oxygen consumption and substrate yield in fermentations. In one aspect, the invention provides a method for regulating the addition of oxygen (O₂) to a fermentor during a fermentation process, comprising measuring real-time dissolved oxygen (DO) in a fermentation broth, measuring real-time O₂ concentration in the fermentor exhaust and providing the real-time DO measurement and real-time O₂ measurement to an adaptive controller configured to regulate O₂ flow into the fermentor responsive to the real-time DO measurement and real-time O₂ measurement.

In another aspect, the invention provides a method for regulating the addition of O₂ in a fermentor during a fermentation process, comprising measuring real-time dissolved oxygen (DO) in a fermentation broth, measuring real-time O₂ concentration in the fermentor exhaust, and providing the real-time DO measurement and real-time O₂ measurement to an adaptive controller, wherein the adaptive controller is configured to regulate incoming O₂ flow and agitation speed in the fermentor responsive to the real-time DO measurement and real-time O₂ measurement.

In another aspect, the invention provides a method for regulating the addition of O₂ to a fermentor during a fermentation process, comprising measuring real-time dissolved oxygen (DO) in a fermentation broth, measuring real-time O₂ concentration in the fermentor exhaust, measuring an additional real-time parameter in the fermentation broth and providing the real-time DO measurement, real-time O₂ measurement, and additional real-time parameter measurement to an adaptive controller, wherein the adaptive controller is configured to regulate incoming O₂ flow and agitation speed in the fermentor responsive to the real-time DO measurement, real-time O₂ measurement and additional real-time parameter measurement.

In another aspect, the invention provides a method for regulating the addition of O₂ to a fermentor during a fermentation process, comprising measuring real-time dissolved oxygen (DO) in a fermentation broth, measuring real-time O₂ concentration in the fermentor exhaust, and providing the real-time DO measurement and real-time O₂ measurement to an adaptive controller configured to regulate O₂ and N₂ flow into the fermentor responsive to the real-time DO measurement and real-time O₂ measurement.

In another aspect, the invention provides a system for regulating addition of O₂ during a fermentation process, comprising a fermentor, a first measuring device configured for measuring real-time dissolved oxygen (DO) in a fermentation broth, a second measuring device configured for measuring real-time O₂ concentration in the fermentor exhaust, and an adaptive controller configured to regulate O₂ flow into the fermentor responsive to the real-time DO measurement and real-time O₂ measurement.

In another aspect, the invention provides a system for regulating addition of O₂ during a fermentation process, comprising a fermentor, a first measuring device configured for measuring real-time dissolved oxygen (DO) in a fermentation broth, a second measuring device configured for measuring real-time O₂ concentration in the fermentor exhaust, and an adaptive controller configured to regulate incoming O₂ flow and agitation speed in the fermentor responsive to the real-time DO measurement and real-time O₂ measurement.

In another aspect, the invention provides a system for regulating addition of O₂ during a fermentation process, comprising a fermentor, a first measuring device configured for measuring real-time dissolved oxygen (DO) in a fermentation broth, a second measuring device configured for measuring real-time O₂ concentration in the fermentor exhaust, a third measuring device configured for measuring an additional real-time parameter in the fermentation broth, and an adaptive controller configured to regulate incoming O₂ flow and agitation speed in the fermentor responsive to the real-time DO measurement, real-time O₂ measurement and additional real-time parameter measurement.

In another aspect, the invention provides a system for regulating addition of O₂ during a fermentation process, comprising a fermentor, a first measuring device configured for measuring real-time dissolved oxygen (DO) in a fermentation broth, a second measuring device configured for measuring real-time O₂ concentration in the fermentor exhaust, and an adaptive controller configured to regulate O₂ and N₂ flow into the fermentor responsive to the real-time DO measurement and real-time O₂ measurement.

### BRIEF DESCRIPTION OF THE DRAWINGS

For a further understanding of the nature and objects of the present invention, reference should be made to the following detailed description, taken in conjunction with the accompanying drawings, in which like elements are given the same or analogous reference numbers and wherein:

FIG. 1 is an embodiment of a process control system with two measurements and one manipulated variable in cascade form.

FIG. 2 is an embodiment of a process control system used to control the O₂ level in a fermentor which is mechanically agitated.

FIG. 3 is an embodiment of a process control block diagram system using a model-adaptive controller.

FIG. 4 is an embodiment of a process control system using an adaptive controller to control the O₂ level in a fermentor.

FIG. 5 is an embodiment of a process control system using an adaptive controller to control the O₂ level and agitation speed in a fermentor.

FIG. 6 is an embodiment of a process control system using an adaptive controller to control the O₂ level and agitation speed in a fermentor, including an additional sensing element for more real-time measurements.

FIG. 7 is an embodiment of a process control system using adaptive controllers to control the O₂ level and N₂ level in a bubble type fermentor.

### DESCRIPTION OF PREFERRED EMBODIMENTS

The words and phrases used herein should be given their ordinary and customary meaning in the art by one skilled in the art unless otherwise further defined.

Aerobic fermentation is an important biochemical process typically conducted in a controlled reaction vessel. The environmental conditions in the vessel, such as gas exhaust content, temperature, pH, dissolved oxygen levels, and agitation speed rate need to be closely monitored and controlled to promote larger cell growth and product formation rates. The presence of pure oxygen in the fermentor promotes a high oxygen transfer rate to the microorganisms, enhancing growth and production formation. Embodiments of the present invention provide a system for manipulating the oxygen flow rate during an aerobic fermentation such that the productivity, yield and the oxygen transfer efficiency are maximized. The embodiments described herein offer methods and systems to accurately calculate the amount of pure oxygen to supply in an agitated fermentor based on real-time measurements of DO and O₂ exhaust concentrations, while maintaining desired agitation speed for desired pH levels or CO₂ removal. In bubble and airlift fermentors, the N₂ supply is controlled in addition to O₂ supply. Illustrative embodiments of the present invention include batch and fed-batch fermentations but other modes of operation are also contemplated.

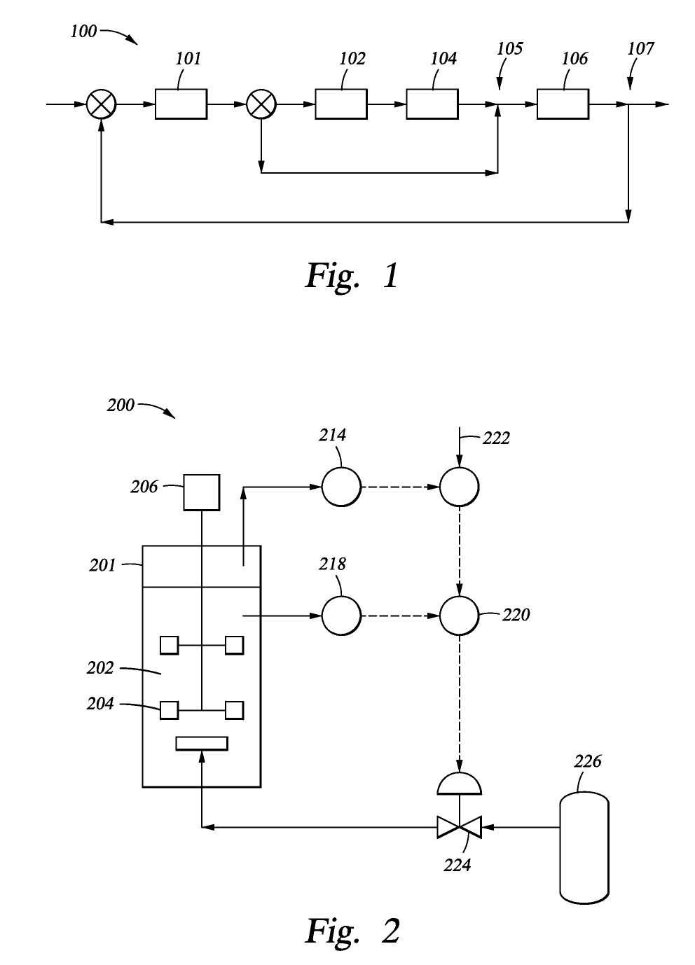

The embodiments of the invention include various process control systems to control the amount of pure oxygen supplied to a fermentor. FIG. 1 shows a process control system 100 using a cascade control. According to one embodiment, a cascade control is the combination of two or more controllers, where an output signal from one controller forms a setpoint of the other. A cascade control is used when there are two or more available measurements, but only one manipulated variable. In the process control system 100, a set of controllers 101 and 102 control a process that is subdivided into two separate processes. The output of process 104 is one process variable that is monitored, and 106 is another process whose output is the controlling variable. A process variable typically entails an intermediate procedure affecting a manipulated variable, or output. For example, in one embodiment, a process variable can involve the opening and closing of an O₂ valve connected to an O₂ source, thereby affecting a manipulated variable, the total O₂ flow rate. The process control system 100 allows the controller 101 to change the set point of the second controller 102. The controller 102 measures one variable 105 and controls process 106 with the final controlling variable 107.

In one embodiment, the process control system 100 described above is applied to a fermentor. FIG. 2 is a diagram showing a process control system 200 using an embodiment of the cascade control system 100 to control the O₂ level in a fermentor 201. The fermentor 201 includes a motor 206 connected to an impeller 204 which affects a desired agitation. In one embodiment, the agitation speed provided by the motor 206 and connected impeller 204 remains constant. The amount of O₂ exhaust in the headspace of the agitation fermentor 201 is measured by an oxygen sensor 214 and relayed to an O₂ controller 222. The controller 222 is programmed to maintain a relatively low O₂ concentration level in the exhaust. The dissolved oxygen (DO) in a broth mixture 202 measured by the DO probe 218 and regulated by a DO controller 220. An output signal from the O₂ controller 222 defines a setpoint for the DO controller 220, which in turn regulates the oxygen flow rate through the opening of an O₂ valve 224 connected to an O₂ source 226.

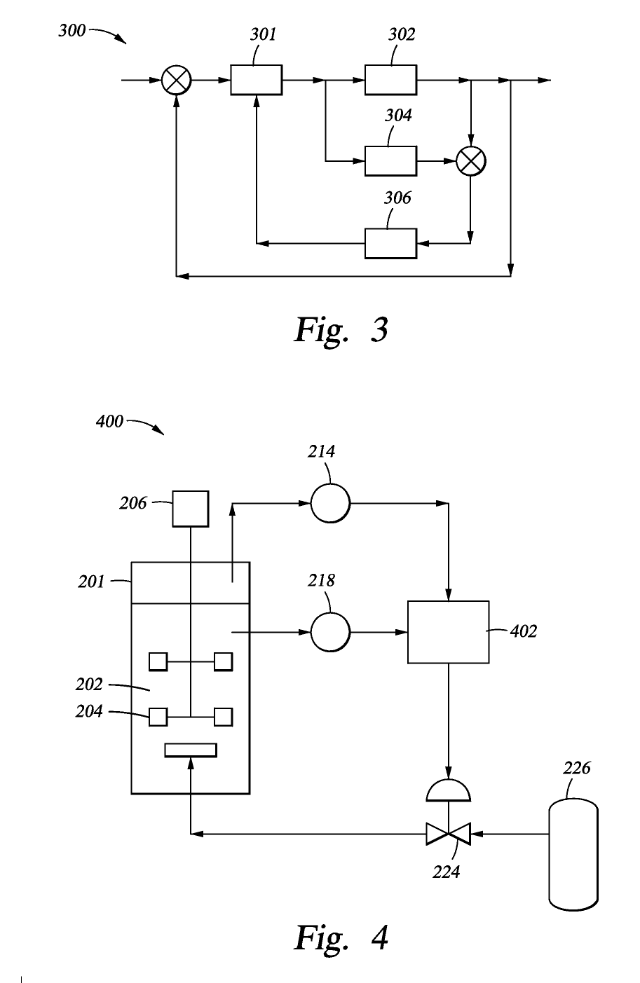

FIG. 3 shows a process control block diagram system 300, according to another embodiment, in which a model-based adaptive controller is used. In one embodiment, the model-based adaptive controller may be applied to an agitated fermentor to control the supply of O₂, as will be described with respect to FIG. 4, below. In this system, an adaptive controller 301 performs according to a specific model of a controlled process. The controller 301 is used to control measured model deviations from the desired process model. In one embodiment of the process control system 300, only one manipulated variable applied to controller 302 is controlled by the adaptive controller 301. An adaptation 306 is made after comparing the measurement of the process 302 to the output of the model of the controlled process 304. The adaptation 306 affects the tuning parameters of the adaptive controller 301, which in turn affects the manipulated variable applied to the process 302.

As noted above, an embodiment of the process control system 300 described above can be applied to a mechanically agitated fermentor. FIG. 4 is a diagram showing a process control system 400 using an embodiment of the model-adaptive control system 300 to control the O₂ level in a mechanically agitated fermentor 201. The adaptive controller 402 uses the real-time measurements from the DO probe 218 and the O₂ exhaust sensor 214. The adaptive controller 402 compares these measurements to a specific process model. In one embodiment, the process model can be the behavior of the DO based on the gas supply. After the comparison with the process model, the adaptive controller 402 regulates the oxygen flow rate through the opening of an O₂ valve 224 connected to an O₂ source 226.

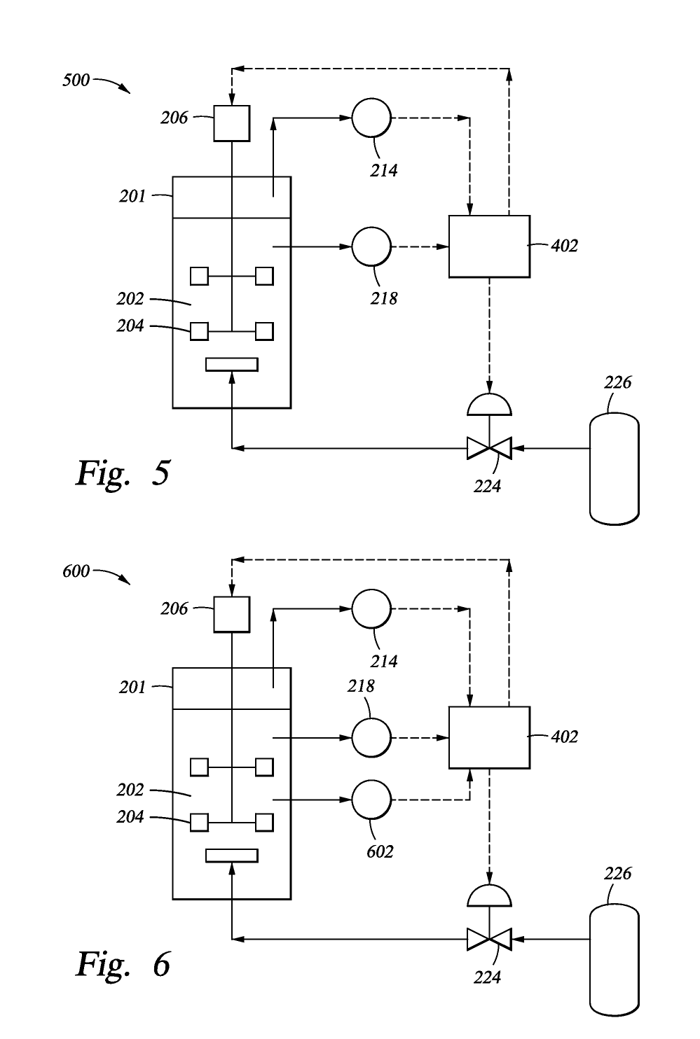

The agitation speed of a rotor inside a fermentor can affect the amount of DO and O₂ exhaust during a fermentation. Accordingly, in another embodiment, the agitation speed of the rotor is controlled. FIG. 5 exhibits an embodiment of a process control system 500 in which the model-adaptive control system 300 controls the O₂ level and agitation speed in a mechanically agitated fermentor 201. The adaptive controller 402 uses the real-time measurements from the DO probe 218 and the O₂ exhaust sensor 214. The adaptive controller 402 compares these measurements to a specific process model, and regulates the oxygen flow rate through the opening of an O₂ valve 224 connected to an O₂ source 226. The adaptive controller 402 also regulates the agitation speed of the motor 206 connected to the agitator 204 in the agitation fermentor 201.

Additional variables such as pH, cell density, and cell product formation can help determine the optimum amount of O₂ to be supplied during a fermentation. FIG. 6 exhibits a process control system 600 using another embodiment of the model-adaptive control system 300 to control the O₂ level in a fermentor 201. The adaptive controller 402 uses the real-time measurements from the DO probe 218, the O₂ exhaust sensor 214, and an additional sensing element 602. The additional sensing element can measure variables in the fermentation broth 202 related the cell mass growth (pH, optical density) or cell products during fermentation. The adaptive controller 402 compares these measurements to a specific process model, and regulates the oxygen flow rate through the opening of an O₂ valve 224 connected to an O₂ source 226. The adaptive controller 402 also regulates the agitation speed of the motor 206 connected to the agitator 204 in the agitation fermentor 201.

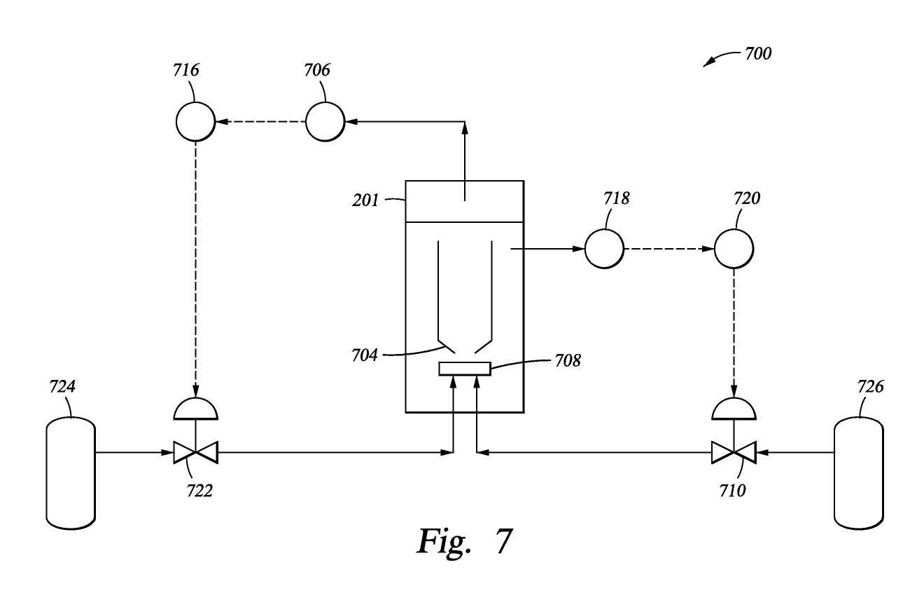

Addition of pure oxygen to bubble type or airlifted fermentors may require the removal of excess CO₂. To accomplish this, an additional injection of N₂ may be utilized to remove CO₂ and provide extra mixing, according to one embodiment. Therefore, the manipulated variables in the bubble fermentor are the O₂ and N₂ flow rates. FIG. 7 is an embodiment showing a process control system 700 having a bubble fermentor 701, in which bubbles generated at a gas injection system 708 provide the agitation. The injector 708 can consist of a gas distribution plate of varying diameter located at the lower section of the bioreactor. The injector 708 is directly connected to the gas sources. In this embodiment, a controller 716 regulates the O₂ flow rates through the opening of an O₂ valve 722 connected to an O₂ source 724. Another controller 720 regulates the N₂ flow rates through the opening of a N₂ valve 710 connected to a N₂ source 726. The controlled variables in this embodiment are DO measured by a DO sensor 718, and O₂ level in the fermentor measured by an O₂ exhaust sensor 706. An inner draft tube 704 prevents the coalescing of bubbles and promotes efficient mixing in the fermentor. An airlift reactor is another possible embodiment similar in design to this figure without the inner draft tube 704.

### Examples

The following example is presented for a further understanding of the nature and objects of the present invention. The example is illustrative only and other embodiments of the integrated processes and apparatus may be employed without departing from the true scope of the invention.

This example describes one model that can be used to control oxygen flow into a fermentor during an aerobic fermentation. The model can consist of following equations, which reflect the most important interactions during the fermentation process when a gas flow rate into the fermentor is controlled by an adaptive controller:

$$\begin{matrix}μ = \frac{{μ}_{m} S}{( {K}_{s} + S )} & ( 1 ) \\ {μ}_{o} = \frac{{μ}_{om} {O}_{2}}{( {K}_{o} + {O}_{2} )} & ( 2 ) \\ \frac{d X}{d t} = ( μ * {μ}_{o} ) X & ( 3 ) \\ \frac{d S}{d t} = - μ X / {Y}_{XS} & ( 4 ) \\ \frac{d P}{d t} = μ X / {Y}_{XP} & ( 5 ) \\ \frac{d {O}_{2}}{d t} = kla ( {O}_{2}^{*} - {O}_{2} ) - {μ}_{o} X / {Y}_{xo} = OTR - OCR & ( 6 ) \\ {F}_{o , exit} = {F}_{i} - OTR & ( 7 ) \\ OTR = kla * 1.15 * {F}_{i} ( {O}_{2}^{*} - {O}_{2} ) & ( 8 ) \\ M ( k ) = M ( k - 1 ) + {b}_{o} E ( k - 1 ) + {b}_{1} ( ( E ( k ) - E ( k - 1 ) ) & ( 9 )\end{matrix}$$

### Nomenclature:

μ Substrate growth rate
μₘ Substrate specific growth rate
S Substrate concentration
Kₛ Substrate inhibition constant
μₒ Oxygen growth rate
μₒₘ Oxygen specific growth rate
O₂ Oxygen concentration
X Cell mass concentration
Yₓₛ Cell mass to substrate yield
P Product concentration
Yₓₚ Cell mass to product yield
kla Mass transfer coefficient
O₂* Equilibrium oxygen concentration in the broth
Yₓₒ Cell mass to oxygen yield
OTR Oxygen transfer rate
OCR Oxygen consumption rate
F_{o,exit} Oxygen flow exiting the fermentor
Fᵢ Oxygen flow entering the fermentor
M Manipulating variable
E Error between setpoint and O₂ in media
b₀ Controller tuning parameter
b₁Controller tuning parameter
k Time interval

Equation 1 represents a typical microorganism growth rate represented by the Monod Equation. The microorganism growth rate can be influenced by O₂ as a substrate in a fermentor, and Equation 1 can be modified to Equation 2 to include the addition of O₂. The overall cell mass concentration can be represented as the multiplicative contribution of the substrate and the oxygen concentrations. Equation 3 represents the change in cell mass concentration as a function of the substrate growth rate and oxygen growth rate. Equation 4 represents the substrate consumption and Equation 5 represents the product formation during a fermentation. Both the substrate consumption and product formation rates are limited by the corresponding yields. Equation 6 reflects the overall O₂ available in the media, which is a function of the oxygen transferred from the gas phase to the media (OTR) and the oxygen consumed by the microorganisms (OCR).

There is a continuous supply and removal of gas from the fermentor in this model. Using a material balance, the amount of gas exiting the fermentor is calculated as the difference of the gas supply and the oxygen transferred to the media (OTR) as shown in Equation 7, the OTR being calculated in Equation 8. In reality, the mass transfer coefficient, kla, is a function of the inlet gas flow rate as it changes the size of gas bubbles. The mass transfer coefficient can also change with time due to physical properties of the media during the fermentation, and an example of a time varying kla is given by Equation 10:

kla=1e−06*t²−0.0001*t+0.0038; (10)

In order to perform a control experiment, a control algorithm is needed. A controller that can be used in this model is known as a proportional plus integral controller (PI). Equation 9 represents a controller in discrete form, in which M is the manipulating variable, in this case the gas inlet flow, Fᵢ, and E is the error between the set point and the controlled variable, the O₂ in the media. The constants bₒ and b₁ are the controller tuning parameters, and k represents the time interval. The parameters in Equation 9 are constant parameters that are satisfactory for processes that do not change significantly with time. However, as shown in Equation 10, fermentation processes can change significantly with time. Therefore, the controller parameters, bₒ and b₁, are not necessarily kept constant. An example of a simple adaptive controller is given by Equation 11, which shows that the controller parameter changes as the O₂ measurement changes:

bo=−21.55*O₂+2.1664 (11)

Processes and apparatus for practicing the present invention have been described. It will be understood and readily apparent to the skilled artisan that many changes and modifications may be made to the above-described embodiments without departing from the spirit and the scope of the present invention. The foregoing is illustrative only and other embodiments of the integrated processes and apparatus may be employed without departing from the true scope of the invention defined in the following claims.

What is claimed is:

1. A method for regulating the addition of oxygen (O₂) to a fermentor during a fermentation process, comprising:
a) measuring real-time dissolved oxygen (DO) in a fermentation broth;
b) measuring real-time O₂ concentration in fermentor exhaust of the fermentor; and
c) providing the real-time DO measurement and real-time O₂ measurement to an adaptive controller configured to regulate O₂ flow into the fermentor responsive to the real-time DO measurement and real-time O₂ measurement.

2. The method of claim 1, wherein the fermentor is mechanically agitated fermentor.

3. The method of claim 1, further comprising a probe in the fermentation broth configured to measure the real-time DO.

4. The method of claim 1, wherein the real-time O₂ concentration in the fermentor exhaust is measured by a sensor in a headspace of the fermentor.

5. The method of claim 1, wherein the adaptive controller regulates O₂ flow into the fermentor by acting on an O₂ control valve connected to an O₂ source.

6. The method of claim 1, wherein the adaptive controller regulates O₂ flow into the fermentor by implementing a model which defines a desired process.

7. A method for regulating the addition of O₂ in a fermentor during a fermentation process, comprising:
a) measuring real-time dissolved oxygen (DO) in a fermentation broth;
b) measuring real-time O₂ concentration in fermentor exhaust of the fermentor; and
c) providing the real-time DO measurement and real-time O₂ measurement to an adaptive controller, wherein the adaptive controller is configured to regulate incoming O₂ flow and agitation speed in the fermentor responsive to the real-time DO measurement and real-time O₂ measurement.

8. The method of claim 7, wherein the fermentor is an mechanically agitated fermentor.

9. The method of claim 7, further comprising a probe in the fermentation broth configured to measure the real-time DO.

10. The method of claim 7, wherein the real-time O₂ concentration in the fermentor exhaust is measured by a sensor in a headspace of the fermentor.

11. The method of claim 7, wherein the adaptive controller regulates O₂ flow into the fermentor by acting on an O₂ control valve connected to an O₂ source.

12. The method of claim 7, wherein the adaptive controller regulates the agitation speed in the fermentor by acting on a motor connected to an agitator in the fermentation broth.

13. The method of claim 7, wherein the adaptive controller regulates O₂ flow into the fermentor by implementing a model which defines a desired process.

14. A method for regulating the addition of O₂ to a fermentor during a fermentation process, comprising:
a) measuring real-time dissolved oxygen (DO) in a fermentation broth;
b) measuring real-time O₂ concentration in fermentor exhaust of the fermentor;
c) measuring an additional real-time parameter in the fermentation broth; and
d) providing the real-time DO measurement, real-time O₂ measurement, and additional real-time parameter measurement to an adaptive controller, wherein the adaptive controller is configured to regulate incoming O₂ flow and agitation speed in the fermentor responsive to the real-time DO measurement, real-time O₂ measurement and additional real-time parameter measurement.

15. The method of claim 14, wherein the fermentor is a mechanically agitated fermentor.

16. The method of claim 14, further comprising a probe in the fermentation broth configured to measure the real-time DO.

17. The method of claim 14, wherein the real-time O₂ concentration in the fermentor exhaust is measured by a sensor in a headspace of the fermentor.

18. The method of claim 14, wherein the additional parameter measured can be cell density in the fermentation broth, pH of the fermentation broth, temperature of the broth, quantity of cellular products in the fermentation broth, or carbon dioxide (CO₂) concentration in the fermentor.

19. The method of claim 14, wherein the adaptive controller regulates O₂ flow into the fermentor by acting on an O₂ control valve connected to an O₂ source.

20. The method of claim 14, wherein the adaptive controller regulates the agitation speed in the fermentor by acting on a motor connected to an agitator in the fermentation broth.

21. The method of claim 14, wherein the adaptive controller regulates O₂ flow into the fermentor by implementing a model which defines a desired process.

22. A method for regulating the addition of O₂ to a fermentor during a fermentation process, comprising
a) measuring real-time dissolved oxygen (DO) in a fermentation broth;
b) measuring real-time O₂ concentration in fermentor exhaust of the fermentor; and
c) providing the real-time DO measurement and real-time O₂ measurement to an adaptive controller configured to regulate O₂ and N₂ flow into the fermentor responsive to the real-time DO measurement and real-time O₂ measurement.

23. The method of claim 22, wherein the fermentor is a bubble fermentor.

24. The method of claim 22, wherein the fermentor is an airlift fermentor.

25. The method of claim 22, further comprising a probe in the fermentation broth configured to measure the real-time DO.

26. The method of claim 22, wherein the real-time O₂ concentration in the fermentor exhaust is measured by a sensor in a headspace of the fermentor.

27. The method of claim 22, wherein the adaptive controller regulates O₂ flow into the fermentor by acting on an O₂ control valve connected to an O₂ source.

28. The method of claim 22, wherein the adaptive controller regulates O₂ flow into the fermentor by implementing a model which defines a desired process.

29. A system for regulating addition of O₂ during a fermentation process, comprising:
a) a fermentor;
b) a first measuring device configured for measuring real-time dissolved oxygen (DO) in a fermentation broth;
c) a second measuring device configured for measuring real-time O₂ concentration in fermentor exhaust of the fermentor; and
d) an adaptive controller configured to regulate O₂ flow into the fermentor responsive to the real-time DO measurement and real-time O₂ measurement.

30. A system for regulating addition of O₂ during a fermentation process, comprising:
a) a fermentor;
b) a first measuring device configured for measuring real-time dissolved oxygen (DO) in a fermentation broth;
c) a second measuring device configured for measuring real-time O₂ concentration in fermentor exhaust of the fermentor; and
d) an adaptive controller configured to regulate incoming O₂ flow and agitation speed in the fermentor responsive to the real-time DO measurement and real-time O₂ measurement.

32. A system for regulating addition of O₂ during a fermentation process, comprising:
a) a fermentor;
b) a first measuring device configured for measuring real-time dissolved oxygen (DO) in a fermentation broth;
c) a second measuring device configured for measuring real-time O₂ concentration in fermentor exhaust of the fermentor;
d) a third measuring device configured for measuring an additional real-time parameter in the fermentation broth; and
e) an adaptive controller configured to regulate incoming O₂ flow and agitation speed in the fermentor responsive to the real-time DO measurement, real-time O₂ measurement and additional real-time parameter measurement.

33. A system for regulating addition of O₂ during a fermentation process, comprising:
a) a fermentor;
b) a first measuring device configured for measuring real-time dissolved oxygen (DO) in a fermentation broth;
c) a second measuring device configured for measuring real-time O₂ concentration in fermentor exhaust of the fermentor; and
d) an adaptive controller configured to regulate O₂ and N₂ flow into the fermentor responsive to the real-time DO measurement and real-time O₂ measurement.
